(19) United States
(12) Patent Application Publication
(10) Pub. No.: US 2004/0243214 A1
(43) Pub. Date: Dec. 2, 2004

(54) Coated stent with protective packaging and method of using same

(75) Inventors: Thomas Farrell, Galway (IE); Colm Quinn, Longford (IE)

Correspondence Address: FRANK C. NICHOLAS, CARDINAL LAW GROUP, Suite 2000, 1603 Orrington Avenue, Evanston, IL, 60201, US

(73) Assignee: Medtronic Vascular, Inc., Santa Rosa, CA

(21) Appl. No.: 10/827,982

(22) Filed: Apr. 20, 2004

Related U.S. Application Data
(60) Provisional application No. 60/464,865, filed on Apr. 23, 2003.

(51) Int. Cl. A61F 2/06; A61J 1/00

(52) U.S. Cl. 623/1.11; 623/1.46; 206/370

(57) ABSTRACT

A coated stent with protective packaging is provided. The coated stent comprises at least one stent segment and a tray including a stent void disposed therein, wherein the stent segment is restricted from movement with respect to the tray while disposed within the stent void. Systems and method for the coated stent are also provided.

### RELATED APPLICATION

This application claims priority to U.S. Provisional Application No. 60/464,865, “Coated Stent with Protective Packaging and Method of Using Same” to Thomas Farrell and Colm Quinn, filed Apr. 23, 2003, the entirety of which is incorporated by reference.

### FIELD OF THE INVENTION

The invention relates to cardiovascular stents, and specifically to a coated stent with protective packaging.

### BACKGROUND OF THE INVENTION

Cardiovascular disease, including atherosclerosis, is the leading cause of death in the U.S. A number of methods and devices for treating coronary heart disease have been developed, some of which are specifically designed to treat the complications resulting from atherosclerosis and other forms of coronary arterial narrowing.

One method for treating atherosclerosis and other forms of coronary narrowing is percutaneous transluminal coronary angioplasty, hereinafter referred to as “angioplasty” or “PTCA”. More than one-third of heart disease patients undergo angioplasty—about 1 million people annually worldwide. Some patients undergo angioplasty repeatedly.

The objective in angioplasty is to enlarge the lumen of the affected coronary artery by radial hydraulic expansion. This is generally accomplished by inflating a balloon within the narrowed lumen of the affected artery. Radial expansion of the coronary artery may occur in several different dimensions, and is related to the nature of the plaque. Soft, fatty plaque deposits are flattened by the balloon, while hardened deposits are cracked and split to enlarge the lumen. The wall of the artery itself may also be stretched as the balloon is inflated.

With simple angioplasty, the balloon is threaded through the artery with a catheter and inflated at the place where the blood vessel is blocked. After the procedure, the balloon is then removed. With simple angioplasty alone, about 40-50 percent of arteries close up again or re-narrow. This narrowing is known as restenosis.

To reduce the risk of restenosis, a stent may also be inserted during angioplasty. The stent may be used to prop open the artery once the balloon is removed. The use of a stent may reduce the risk of restenosis to 20-30 percent. The stent is designed to support plaque damaged arterial walls after a blockage has been removed.

Typically, if restenosis occurs with a stent, the doctors may insert highly radioactive pellets into the artery to help prevent further clogging. This radiation therapy can halve the risk of restenosis but presents all the risks associated with radiation therapy.

Restenosis occurs because the blood vessel wall is injured when the stent is implanted. The area then becomes inflamed and new cells form scar tissue. The arterial walls may become so thick in some instances that they protrude into the mesh of the stent. In such cases, a further angioplasty may be undergone, and a new stent may be placed inside the existing one. If restenosis continues, the eventual alternative may then be bypass surgery.

Alternatively, a coated stent may be inserted during the angioplasty. Such a coated stent may eliminate the need for repeat angioplasties and could spare some patients the trauma, risk and prolonged recovery associated with heart bypass surgery.

The coated stent may be coated, for example, with Rapamune, generically known as sirolimus or rapamycin. This drug is used to prevent organ rejection in kidney transplants. It stops new cells from forming without impairing the healing of the vessel. It also dampens inflammation and has antibiotic properties.

In clinical studies, patients who have received the coated stent do not show this re-narrowing and re-blockage of arteries.

However, because the coating of the stent comprises a therapeutic drug, coated stents present problems associated with drug administration. For example, for a drug to be administered effectively, the integrity of the drug's effective dosage should be maintained. Additionally, contamination of the drug should be avoided. Certain drugs also require regulated conditions for efficacy, such as regulated air circulation or lack thereof, regulated exposure to light, etc. Furthermore, some processes in preparing the stent for use, such as sterilization, may affect the drug's efficacy.

Currently, stents are packaged in a sterile flat tray. The stent and balloon catheter are coiled and the stent is secured between the tray and a peel away top. With a coated stent, the tray may damage the coating while the stent is being placed on the tray. Meanwhile, the peel away top may stick to the coating and when the top is removed, the coating may be removed with the top rather than remaining on the stent. Additionally, the stent may move within the package during handling. With such movement, some of the coating may be disturbed. In any of these cases, the coating of the stent may be damaged and the dosage of the drug may be reduced.

Moreover, current packaging for stents does not provide for regulation of ambient conditions such as circulation of air or exposure to light. Without such appropriate regulation, the efficacy of the drug may be reduced.

It would be desirable therefore to provide a packaging assembly for a coated stent that overcomes the above.

### SUMMARY OF THE INVENTION

One aspect of the present invention provides a coated stent with protective packaging. The coated stent comprises at least one stent segment and a tray including a stent void disposed therein, wherein the stent segment is restricted from movement with respect to the tray while disposed within the stent void. The coated stent may also comprise a stylet disposed within the tray proximate the stent void to suspend the at least one stent segment within the stent void. In addition, the stent may comprise at least one first attachment disposed within the tray distal to the stent void, to retain the stent segment within the stent void and at least one second attachment disposed within the tray proximal to the stent void to retain the stent segment within the stent void. In some embodiments of the invention, a coating may be dispersed on the at least one stent segment, and the coating may be any one or more of the following: thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti secretory agents, actin inhibitors, remodeling inhibitors, antisense nucleotides, anti metabolites, antiproliferatives, anticancer chemotherapeutic agents, anti-inflammatory steroid or non-steroidal anti-inflammatory agents, immunosuppressive agents, growth hormone antagonists, growth factors, dopamine agonists, radiotherapeutic agents, peptides, proteins, enzymes, extracellular matrix components, inhibitors, free radical scavengers, chelators, antioxidants, antipolymerases, antiviral agents, photodynamic therapy agents, gene therapy agents, and conjugates thereof.

The coated stent may also comprise a catheter attached to the stent segment, an expandable balloon portion attached to the balloon catheter, wherein the expandable balloon portion expands an inner lumen of the stent segment and a catheter void disposed within the tray, wherein the catheter void holds the catheter. In some embodiments of the invention, the stent may also comprise at least one catheter attachment disposed proximate the catheter void, wherein the catheter attachment retains the catheter within the catheter void.

The coated stent may also include an introducer to receive the stent segment and an introducer void disposed within the tray to hold the introducer.

In addition, the coated stent may include at least one accessory void disposed within the tray to hold an accessory such as desiccant packets, oxygen absorber packets, chemical packets, catheters, guidewires, cannulas and stylets. The coated stent may also include a lid.

Another aspect of the present invention provides a system for treating heart disease comprising a stent coupled to a catheter, the stent including a drug polymer coating and a tray including a stent void disposed therein, wherein the stent is restricted from movement in relation to the tray when the stent is placed in the stent void. The system may also include a stylet disposed proximate the stent void, wherein the stylet suspends the stent within the stent void.

In addition, the system may include at least one first attachment disposed distal to the stent void to retain the stent segment within the stent void and at least one second attachment disposed proximal to the stent void, wherein the second attachment retains the stent segment within the stent void.

In some embodiments of the invention, system includes an expandable balloon portion attached to the catheter and may also include a catheter void to hold the catheter within the tray. The system may also include an introducer to receive the stent segment and an introducer void to hold an introducer within the tray. The system may further include an accessory such as desiccant packets, oxygen absorber packets, chemical packets, catheters, guidewires, cannulas and stylets and at least one accessory void to hold at least one accessory within the tray. The system may also include a lid to seal the tray.

Another aspect of the present invention provides a method of protecting a coated stent. A tray is provided, including a stent void therein. The coated stent is suspended within the stent void such that the coated stent is restricted from movement with respect to the tray when placed in the stent void.

The foregoing, and other, features and advantages of the invention will become further apparent from the following detailed description of the presently preferred embodiments, read in conjunction with the accompanying drawings. The detailed description and drawings are merely illustrative of the invention rather than limiting, the scope of the invention being defined by the appended claims in equivalence thereof.

### BRIEF DESCRIPTION OF THE DRAWINGS

FIG. 1 is a schematic view of one embodiment of a coated stent with protective packaging in accordance with the present invention;

FIG. 2 is a cross-section of the embodiment of the coated stent with protective packaging of FIG. 1;

FIG. 3 is a schematic view of one embodiment of a lid for the coated stent with protective packaging system of FIG. 1; and

FIG. 4 is a schematic view of one embodiment of a coated stent in accordance with the present invention.

### DETAILED DESCRIPTION OF THE PRESENTLY PREFERRED EMBODIMENTS

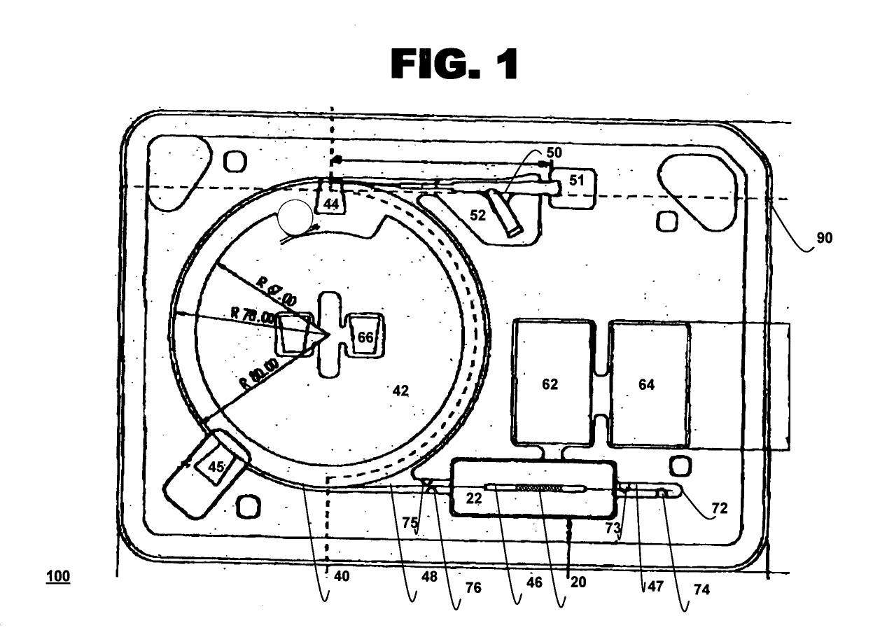

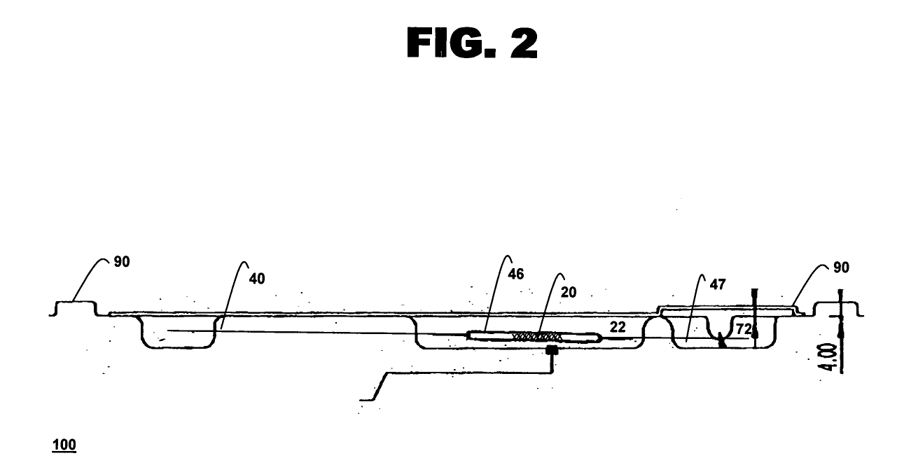

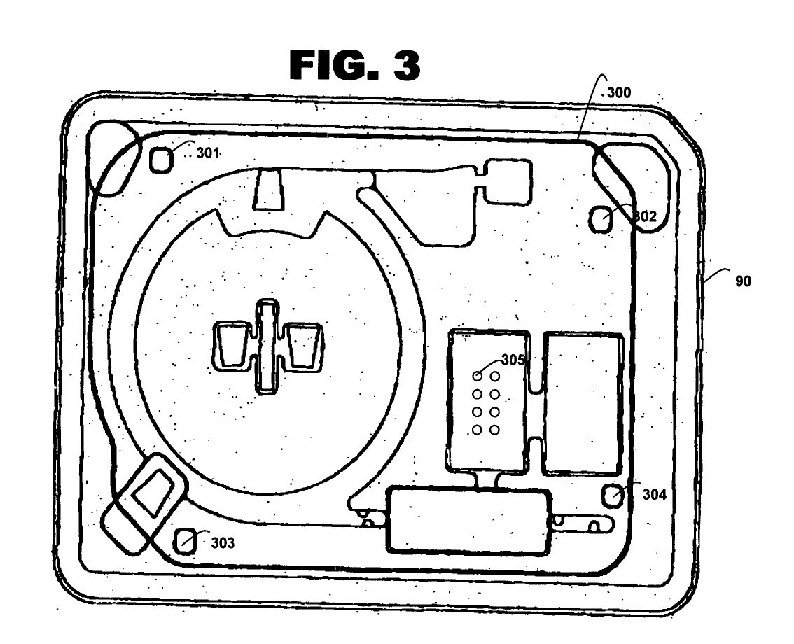

FIG. 1 shows one embodiment of a packaging system in accordance with the present invention at 100. Packaging system 100 may include the packaging tray 90 of FIG. 1 and may further include a lid 300 as seen in FIGS. 2 and 3. Packaging system 100 may comprise a stent 20 disposed within a void 22 of packaging tray 90. As seen in FIG. 2, in one embodiment of the invention, a coating 30 may be dispersed on stent 20. Packaging tray 90 may further comprise additional voids for other components of a stent assembly, including introducer void 52 in which an introducer assembly 50 is disposed. In the embodiment shown in FIG. 1, the stent 20 may be disposed upon a balloon catheter 40. Balloon catheter 40 may further comprise an expandable balloon portion 46. Packaging system 90 may further comprise a stylet 47, which is removably positioned within stylet void 72. Stylet 47 may be held in stylet void 72 by attachments 73, 74 and stent 20 may be suspended upon stylet 47. Thus, in one embodiment of the invention, stent 20 is suspended at two ends, using attachments 73, 74 at one end and attachments 75, 76 at the other end.

In one embodiment of the invention, a proximal portion 48 of balloon catheter 40, which is proximal to stent 20 may be held fast by attachments 75, 76. Because stent 20 is distally suspended on stylet 47 and the balloon catheter 40 on which stent 20 rests is proximally suspended at a portion 48 of balloon catheter 40, stent 20 is suspended within void 22. Stent 20 may thus be prevented from being jostled during shipment and storage. Damage of the coating 30 dispersed on the stent 20 may thus be avoided. All or part of the remaining proximal portion of balloon catheter 40 may be disposed in a catheter void 42 of packaging tray 90. In the embodiment shown in FIG. 1, the shaft of balloon catheter 40 may be looped in tray 90 using one or more loop assemblies 44, 45. Packaging tray 90 may further comprise one or more voids 62, 64, 66 which may be used to hold other materials and accessories, including, but not limited to, dessicants, O₂ absorbers, other chemicals, extra looping assemblies and flushing cannulas.

In other embodiments of the invention, without a balloon catheter, stylet 47 may extend from stylet void 72 through void 22. Stylet 47 may be held by attachments 73, 74 at an end distal to stent 20 and held by attachments 75, 76 at an end proximal to stent 20 and stent 20 may be suspended, without a catheter, on stylet 47.

The stent void 22, introducer void 52, stylet void 72 and additional voids 62, 64, 66 of the present invention may be any suitable formation disposed within, formed as part of or attached to packaging tray 90 including but not limited to a well of packaging tray 90, an indentation in packaging tray 90, a space in packaging tray 90 or a hole in packaging tray 90. Packaging system 100 may also comprise a plurality of voids of all shapes, sizes and formations. For example, packaging system 100 may comprise a stent void 22 that is an indentation formed within packaging tray 90 and an introducer void 52 that is a well attached to packaging tray 90.

FIG. 2 shows a cross-section of packaging system 100. As seen in FIG. 2, lid 300 may lie flat upon tray 90. Stent 20 may be disposed within a void 22 of packaging tray 90. In one embodiment of the invention, a coating 30 may be dispersed on stent 20. Packaging tray 90 may further comprise additional voids for other components of a stent assembly, such as void 46 in which a loop assembly is disposed for holding balloon catheter 40, upon which stent 20 may be disposed. Balloon catheter 40 may further comprise an expandable balloon portion 45. Packaging system 100 may further comprise a stylet 47, which is removably positioned within void 72. Stylet 47 may be held in stylet void 72 by attachments 73, 74 and stent 20 may be suspended upon stylet 47.

Stylet 47 may be any suitable assembly that enables suspension of stent 20 in void 22. For example, stylet 47 may be an extended portion of balloon catheter 40 that is distal to stent 20. Alternatively, stylet 47 may be an assembly that is insertable into the lumen of stent 20 and that may be attached with attachments 73, 74 in such a manner as to suspend stent 20 in void 22. For example, stylet 47 may be a thin needle or wire upon which stent 20 may be suspended. Alternatively, stylet 47 may be an assembly formed within tray 90. For example, stylet 47 may be a thin column formed within void 22 that has an “eye” through which an end of catheter 40 may be threaded such that stent 20 is suspended within void 22. Stylet 47 may be an assembly that is removable with stent 20 from tray 90. For example, stylet 47 may be a modified extension of catheter 40. Alternatively, stylet 47 may remain in tray 90 once stent 20 is removed.

In some embodiments of the invention, stylet void 72 of packaging tray 90 is sized and adapted so that all or a portion of stylet 47 may be held within void 72. Stylet void 72 may be any suitably sized space or pocket for holding stylet 47. For example, in one embodiment of the invention, stylet void 72 is a narrow channel into which stylet 47 protrudes. Stylet void 72 may be formed within packaging tray 90 using any suitable means, such as for example, thermomolding stylet void 72 within tray 90 when tray 90 is being thermoformed. Alternatively, tray 90 may be formed first and stylet void 72 inserted into the tray 90 afterwards.

Stylet void 72 may further comprise one or more attachment portions 73, 74. These attachment portions 7374 attach stylet 47 within stylet void 72. As seen in FIG. 1, attachment portions 73, 74 may be formed bumps along the sides of stylet void 72 that hold stylet 47 within stylet void 72. Attachment portions 73, 74 may be thermoformed within packaging tray 90. Alternatively, attachment portions 73, 74 may be inserted into tray 90 after the tray has been formed. For example, attachment portions 73, 74 may be adhesive segments that adhere to stylet 47 to hold it within stylet void 72.

In some embodiments of the invention, void 22 of packaging tray 90 is sized and adapted so that all or a portion of stent 20 may be suspended within void 22. Void 22 may be any suitably sized space or pocket in which a stent 20 may be suspended. For example, in one embodiment of the invention, void 22 is a well of packaging tray 90 that is 80 mm in length by 27 mm in width by 13 mm in depth. Void 22 may be formed within packaging tray 90 using any suitable means, such as for example, thermomolding void 22 within tray 90 when tray 90 is being thermoformed. Alternatively, tray 90 may be formed first and void 22 inserted into the tray 90 afterwards.

Packaging tray 90 may further comprise additional voids for other components of a stent assembly, including introducer void 52 in which an introducer assembly 50 is disposed. In one embodiment of the invention, introducer void 52 is sized and adapted so that all or a portion of introducer assembly 50 may be placed into introducer void 52. Alternatively, as seen in FIG. 1, packaging tray 90 may include a void 51 into which a portion of introducer assembly 50 may be introduced to suspend introducer assembly 50 in introducer void 52. For example, a lure component of introducer assembly 50 may snap into void 51 to hold assembly 50 in place. Introducer void 52 may be any suitably sized space or pocket in which an introducer assembly 50 may be placed. For example, in one embodiment of the invention, introducer void 52 mimics the shape of an introducer assembly 50. Introducer void 52 may be formed within packaging tray 90 using any suitable means, such as for example, thermomolding introducer void 52 within tray 90 when tray 90 is being thermoformed. Alternatively, tray 90 may be formed first and introducer void 52 inserted into the tray 90 afterwards.

Introducer assembly 50 may be used to introduce stent 20 via catheter 40 to the desired site. Introducer assembly 50 may be any suitable introducer assembly as is well known in the art. For example, introducer assembly 50 may be a conventional guide wire assembly.

Catheter 40 may be, for example, a low profile design with a tapered distal tip, and an inner lumen for insertion of a conventional guide wire. Any conventional or modified balloon catheter device may be used, such as a PTCA balloon catheter. In one embodiment of the invention, one or more guide wires may be stored in one or more of voids 62, 64, 66 of packaging tray 90.

The expandable balloon portion 46 may be formed from a material such as polyethylene, polyethylene terephthalate (PET), or from nylon or the like. The length and diameter of the balloon may be selected to accommodate the particular configuration of the stent segment 20. The balloon may be carried on any catheter, such as, for example PTCA low profile catheters and over the wire catheters.

In some embodiments of the invention, as described above, balloon catheter 40 may further comprise a stylet 47, which protrudes into void 72. Tip portion 47 of balloon catheter 40 may be held in stylet void 72 by attachments 73, 74 while another portion 48 of balloon catheter 40 distal to stent 20 may be held fast by attachments 75, 76. Because tip portion 47 is held by attachments 73, 74 and distal portion 48 is held by attachments 75, 76, stent 20 is suspended within void 22. Stent 20 may thus be prevented from moving and damage of the coating dispersed on the stent may be avoided. All or part of the remaining distal portion of balloon catheter 40 may be disposed in a catheter void 42 of packaging tray 90. In the embodiment shown in FIG. 1, the shaft of balloon catheter 40 may be held in catheter void 42 of tray 90 using one or more loop assemblies 44, 45.

In some embodiments of the invention, catheter void 42 of packaging tray 90 is sized and adapted so that all or a portion of catheter 40 may be contained within. Catheter void 42 may be any suitably sized space or pocket for containing a catheter 40. For example, in one embodiment of the invention, void 42 is a circular well of packaging tray 90. Catheter void 42 may be formed within packaging tray 90 using any suitable means, such as for example, thermomolding void 42 within tray 90 when tray 90 is being thermoformed. Alternatively, tray 90 may be formed first and catheter void 42 inserted into the tray 90 afterwards.

Catheter void 42 may further comprise one or more attachment portions 75, 76. These attachment portions 75, 76 attach all or a portion of catheter 40 within catheter void 42. As seen in FIG. 1, attachment portions 75, 76 may be formed bumps along the walls of catheter void 42 that hold catheter 40 within catheter void 42. Attachment portions 75, 76 may be thermoformed within packaging tray 90. Alternatively, attachment portions 75, 76 may be inserted into tray 90 after the tray has been formed. For example, attachment portions 75, 76 may be adhesive segments that adhere to catheter 40 to hold it within catheter void 42.

In one embodiment of the invention, tray 90 is fabricated of clear material to allow for viewing. Alternatively, the tray 90 may be fabricated of opaque material to aid in preventing degradation of the coating on stent 20 from light. The tray 90 may be made, for example, of a high barrier plastic, or other suitable materials. Tray 90 may be thermoformed, for example, from polyethylene, or another suitable material.

Packaging tray 90 may further comprise one or more voids 62, 64, 66 which may be used to hold other materials and accessories. For example, a coated stent may be more sensitive to moisture. One or more of voids 62, 64, 66 may therefore be used to hold desiccant packets or oxygen absorber packets. Voids 62, 64, 66 may also hold any suitable chemicals that may be used to prolong shelf-life or prevent decay of coating 30 on stent 20. Additional accessories may also be stored within one or more of voids 62, 64, 66 including, but not limited to, additional catheters, additional guidewires, additional introducer assemblies, flushing cannulas and additional stylets.

In some embodiments of the invention, voids 62, 64, 66 of packaging tray 90 are of varying sizes depending on what may be contained within each void. Each void 62, 64, 66 may also be individually adapted depending on its contents. Voids 62, 64, 66 may be formed within packaging tray 90 using any suitable means, such as for example, thermomolding voids 62, 64, 66 within tray 90 when tray 90 is being thermoformed. Alternatively, tray 90 may be formed first and voids 62, 64, 66 inserted into the tray 90 afterwards.

FIG. 3 shows lid 300 of packaging system 100. In one embodiment of the invention, lid 300 is fabricated of clear material to allow for viewing. Alternatively, the lid 300 may be fabricated of opaque material to aid in preventing degradation of the coating 30 on stent 20 from light. The lid 300 may be made, for example, of a high barrier plastic, or other suitable materials. In some embodiments of the invention, tray 90 and lid 300 may be made of different materials. For example, tray 90 may be made of a high barrier plastic and lid 300 may be a foil cover over tray 90 or a foil pouch into which tray 90 may be sealed. Lid 300 may be made of foil, for example, in order to prevent moisture and oxygen from affecting components packaged in tray 90. Alternatively, lid 300 may be made from the same material as tray 90. For example, lid 300 and tray 90 may be formed of the same thermoformed plastic, such as polyethylene. In the embodiment of FIG. 3, lid 300 may fit into one or more indentations 301, 302, 303, 304. Lid 300 may be raised over void 22 that contains coated stent 20. Thus, lid 300 is prevented from contacting the coating 30 of stent 20. Lid 300 may further comprise one or more openings 305. Openings 305 may allow air circulation within packaging system 100.

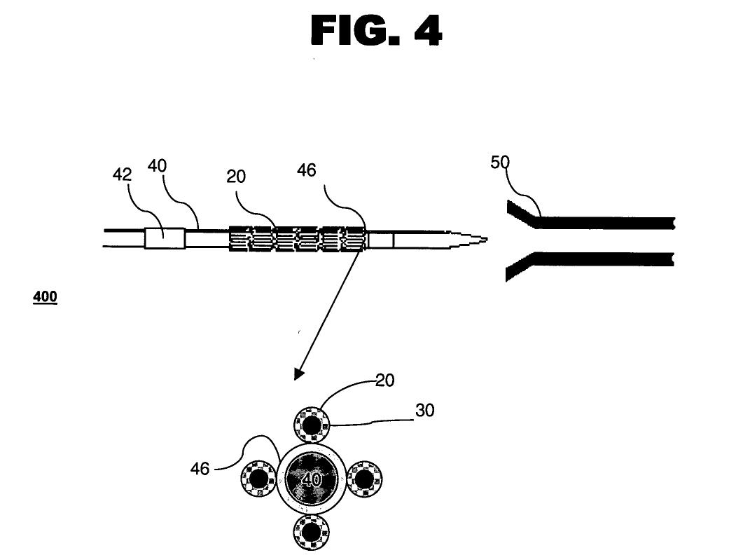

FIG. 4 shows one embodiment of a coated stent for use with packaging system 100 at 400. In the embodiment shown in FIG. 4, one stent segment 20 is shown. However more stent segments 20 may be used depending upon the size and configuration of the narrowed vessel to be treated. Additionally, when more than one stent segment 20 is used, the segments may be connected together by articulated or rigid joints, or multiple single stent segments may be deployed on the balloon catheter 20. When more than one stent segment 20 is deployed on the catheter 40, each segment may have an associated void 22. Alternatively, a plurality of stent segments 20 may be disposed within void 22.

Stent segment 20 may be any suitable device for mechanically keeping an effective blood vessel open after completion of the angioplasty procedure. Such mechanical endoprosthetic devices, which are generally referred to as stents, are typically inserted into the vessel, positioned across the lesion, and then expanded to keep the passageway clear. Stent 20 may be for example, any stent known in the art, including, but not limited to, a coronary stent such as that sold by Medtronic as the S7 system. For example, stent segment 20 may be a self-expanding and expandable stent as is known in the art. Stent segment 20 may be a tubular slotted stents.

As seen in FIG. 4, some embodiments of the invention may include stent retainer rings 42 at one or both ends of the stent 20 to help to maintain the stent on the balloon. These retainers 42 may be located at the proximal and/or distal end of the balloon. Such retainers may be located on top of the balloon 46 or within the balloon 46. Additionally, the balloon portion 46 itself may be used to form one or more stent retainers during encapsulation. Retainers 42 may assist in delivery by providing a smooth transition between the encapsulated stent and the catheter surface. In some embodiments of the invention, retainer rings 42 may interface with stent void 22 in order to hold stent 20 within the void 22.

As seen in FIG. 4, coating 30 may comprise any suitable therapeutic agent for delivering therapy to a target site and/or any suitable substance within which such therapeutic agents may be dispersed. Coating 30 may be a coating adapted to deliver sustained release of therapeutic agent to target cells. Coating 30 may be, for example a biodegradable coating or a porous non-biodegradable coating, having dispersed therein a sustained-release dosage form of one or more therapeutic agents as described below. In an alternative embodiment, a biodegradable stent may also have the therapeutic agent impregnated therein, i.e., within the stent matrix of stent segment 20. In yet another embodiment of the invention, the therapeutic agent(s) may be impregnated within stent segment 20, which is further coated with a coating 30 having the sustained release-dosage form dispersed therein, is also contemplated. This embodiment of the invention would provide a differential release rate of the therapeutic agent, i.e., there would be a faster release of the therapeutic agent from the coating 30 followed by delayed release of the therapeutic agent that was impregnated in the stent matrix upon degradation of the stent matrix. The stent segment 20 may thus provide a mechanical means of increasing luminal area of a vessel, in addition to providing biological stenting action from the therapeutic agents releasably embedded therein.

Coating 30 may take the form of, for example non-degradable microparticulates or nanoparticulates or biodegradable microparticulates or nanoparticulates. The microparticles or nanoparticles may be formed of a polymer-containing matrix that biodegrades by random, nonenzymatic, hydrolytic scission. One embodiment of coating 30 is formed of a mixture of thermoplastic polyesters (e.g., polylactide or polyglycolide) or a copolymer of lactide and glycolide components. The lactide/glycolide structure has the added advantage that biodegradation thereof forms lactic acid and glycolic acid, both normal metabolic products of mammals.

Coating 30 may also be, or may comprise a therapeutic substance which inhibits cellular activity at a target site in order to reduce, delay, or eliminate stenosis after angioplasty or other vascular surgical procedures. Coating 30 may also be a conjugate of several therapeutic substances. For example, coating 30 may comprise therapeutic agents that alter cellular metabolism or are inhibitors of protein synthesis, cellular proliferation, or cell migration; therapeutic agents that affect morphology or increases in cell volume; and/or therapeutic agents that inhibit extracellular matrix synthesis or secretion.

In one embodiment, coating 30 may include a non-cytotoxic therapeutic agent such as, for example, an antisense compound. One example of a non-cytotoxic therapeutic agent is NeuGene® antisense compound, Resten-NG (AVI-4126). Such antisense compounds compete at the mRNA level to block transcription of proteins that are involved in proliferation of the cells that cause restenosis. Antisense compounds may significantly reduce restenosis without prolonging healing times.

In one embodiment, coating 30 may include a cytotoxic therapeutic agent that is a sesquiterpenoid mycotoxin such as a verrucarin or a roridin. Coating 30 may also comprise cytostatic therapeutic agents that inhibit DNA synthesis and proliferation at doses that have a minimal effect on protein synthesis such as protein kinase inhibitors (e.g., staurosporin), suramin, and nitric oxide releasing compounds (e.g., nitroglycerin) or analogs or functional equivalents thereof. In addition, coating 30 may also comprise therapeutic agents that inhibit the contraction or migration of smooth muscle cells and maintain an enlarged luminal area following, for example, angioplasty trauma (e.g., the cytochalasins, such as cytochalasin B, cytochalasin C, cytochalasin D or the like). Coating 30 may also comprise vascular smooth muscle binding proteins that specifically associate with a chondroitin sulfate proteoglycan (CSPG) expressed on the membranes of a vascular smooth muscle cell.

In one embodiment of the invention, coating 30 may comprise agents that exhibit inhibition of a therapeutically significant target cell activity without killing the target cell, or target cell killing activity. For treatment of restenosis of vascular smooth muscle cells, useful therapeutic agents inhibit target cell activity (e.g., proliferation or migration) without killing the target cells. Example therapeutic moieties for this purpose are protein kinase inhibitors (e.g., staurosporin or the like), smooth muscle migration and/or contraction inhibitors (e.g., the cytochalasins, such as cytochalasin B, cytochalasin C, cytochalasin D or the like), suramin, and nitric oxide-releasing compounds, such as nitroglycerin, or analogs or functional equivalents thereof. In cancer therapy, useful therapeutic agents inhibit proliferation or are cytotoxic to the target cells. Example therapeutic moieties for this purpose are Roridin A and Pseudomonas exotoxin, or analogs or functional equivalents thereof. For treatment of immune system-modulated diseases, such as arthritis, useful therapeutic agents deliver cytostatic, cytocidal or metabolism-modulating therapeutic agents to target cells that are accessible by local administration of the dosage form. Example therapeutic moieties for this purpose are Roridin A, Pseudomonas exotoxin, suramin and protein kinase inhibitors (e.g., staurosporin), sphingosine, or analogs or functional equivalents thereof. For treatment of pathologically proliferating normal tissues (e.g., proliferative vitreoretinopathy, corneal pannus and the like), anti-proliferative agents or antimigration agents may be used (e.g., cytochalasins, taxol, somatostatin, somatostatin analogs, N-ethylmaleimide, antisense oligonucleotides and the like).

Other examples of therapeutic agents that may be used alone or in combination within coating 30 include thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti-secretory agents, actin inhibitors, remodeling inhibitors, antisense nucleotides, anti metabolites, antiproliferatives, anticancer chemotherapeutic agents, anti-inflammatory steroid or non-steroidal anti-inflammatory agents, immunosuppressive agents, growth hormone antagonists, growth factors, dopamine agonists, radiotherapeutic agents, peptides, proteins, enzymes, extracellular matrix components, inhibitors, free radical scavengers, chelators, antioxidants, antipolymerases, antiviral agents, photodynamic therapy agents, and gene therapy agents.

The dosage of therapeutic agents may be varied depending on the body lumen involved, the result desired, and the therapy indicated. Preferable therapeutic agents are dispersed within the microparticulates or nanoparticulates of coating 30.

The dosage forms of coating 30 may be targeted to a relevant target cell population by a binding protein or peptide. These binding proteins/peptides may be, for example vascular smooth muscle cell binding protein, tumor cell binding protein and immune system effector cell binding protein. Other possible binding peptides include those that localize to intercellular stroma and matrix located between and among vascular smooth muscle cells. Peptides of this type are specifically associated with collagen, reticulum fibers or other intercellular matrix compounds. Tumor cell binding proteins associated with surface cell markers expressed by the target tumor cell population or cytoplasmic epitopes thereof may also be targeted by the present invention. Immune system-modulated target cell binding proteins associated with cell surface markers of the target immune system effector cells or cytoplasmic epitopes thereof may also be targeted with the present invention. The present invention may also be targeted to pathologically proliferating normal tissues.

Once the components held within packaging system 100 are removed from tray 90, they may be used in any suitable manner known in the art. For example, stent 20 may be delivered to a desired treatment site with or without a guiding catheter and using a conventional guidewire for steerability to negotiate the area to be treated. Conventional radiopaque markers and fluoroscopy may be used with the device for positioning the stent and for viewing the expansion procedure. Once the stent is in place at the treatment site, the balloon portion 46 may be inflated in a conventional manner.

While the primary application for the stent is presently believed to be treatment of cardiovascular disease such as atherosclerosis or other forms of coronary narrowing, the stent assembly of the present invention may also be used for treatment of vessels in the kidney, leg, carotid, or elsewhere in the body. In such other vessels, the size of the stent may need to be adjusted to compensate for the differing sizes of the vessel to be treated.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

We claim:

1. A coated stent with protective packaging, comprising:
At least one stent segment; and
a tray, including a stent void disposed therein, wherein the stent segment is restricted from movement with respect to the tray while disposed within the stent void.

2. The stent of claim 1, further comprising:
a stylet disposed within the tray proximate the stent void to suspend the at least one stent segment within the stent void.

3. The stent of claim 1, further comprising:
at least one first attachment disposed within the tray proximate the stent void and distal to the stent void, wherein the first attachment retains the stent segment within the stent void.

4. The stent of claim 3, further comprising:
at least one second attachment disposed within the tray proximate the stent void and proximal to the stent void, wherein the second attachment retains the stent segment within the stent void.

5. The stent of claim 1, further comprising:
a coating dispersed on the at least one stent segment.

6. The stent of claim 5, wherein the coating is selected from the group consisting of:
thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti secretory agents, actin inhibitors, remodeling inhibitors, antisense nucleotides, anti metabolites, antiproliferatives, anticancer chemotherapeutic agents, anti-inflammatory steroid or non-steroidal anti-inflammatory agents, immunosuppressive agents, growth hormone antagonists, growth factors, dopamine agonists, radiotherapeutic agents, peptides, proteins, enzymes, extracellular matrix components, inhibitors, free radical scavengers, chelators, antioxidants, antipolymerases, antiviral agents, photodynamic therapy agents, gene therapy agents, and conjugates thereof.

7. The stent of claim 1, further comprising:
a catheter attached to the stent segment.

8. The stent of claim 7, further comprising:
an expandable balloon portion attached to the balloon catheter, wherein the expandable balloon portion expands an inner lumen of the stent segment.

9. The stent of claim 7, further comprising:
a catheter void disposed within the tray, wherein the catheter void holds the catheter.

10. The stent of claim 9, further comprising:
at least one catheter attachment disposed proximate the catheter void, wherein the catheter attachment retains the catheter within the catheter void.

11. The stent of claim 1, further comprising:
an introducer to receive the stent segment.

12. The stent of claim 11, further comprising:
an introducer void disposed within the tray wherein the introducer void holds the introducer.

13. The stent of claim 1, further comprising:
at least one accessory void disposed within the tray to hold an accessory.

14. The stent of claim 13 wherein the accessory is selected from the group consisting of:
desiccant packets, oxygen absorber packets, chemical packets, catheters, guidewires, cannulas and stylets.

15. The stent of claim 1, further comprising:
a lid, wherein the lid connects to the tray.

16. A system for treating heart disease, comprising:
a catheter;
a stent coupled to the catheter, the stent including a drug polymer coating; and
a tray including a stent void disposed therein, wherein the stent is restricted from movement in relation to the tray when the stent is placed in the stent void.

17. The system of claim 16, further comprising:
a stylet disposed proximate the stent void, wherein the stylet suspends the stent within the stent void.

18. The system of claim 17, further comprising:
at least one first attachment disposed proximate and distal to the stent void, wherein the first attachment retains the stent segment within the stent void.

19. The system of claim 18, further comprising:
at least one second attachment disposed proximate and proximal to the stent void, wherein the second attachment retains the stent segment within the stent void.

20. The system of claim 16, further comprising:
an expandable balloon portion attached to the catheter.

21. The system of claim 16, further comprising:
a catheter void to hold the catheter within the tray.

22. The system of claim 16, further comprising:
an introducer to receive the stent segment.

23. The system of claim 16, further comprising:
an introducer void to hold an introducer within the tray.

24. The system of claim 16, further comprising at least one accessory selected from the group consisting of:
desiccant packets, oxygen absorber packets, chemical packets, catheters, guidewires, cannulas and stylets.

25. The system of claim 16, further comprising:
at least one accessory void to hold at least one accessory within the tray.

26. The system of claim 16, further comprising:
a lid to seal the tray.

27. A method of protecting a coated stent, comprising:
providing a tray including a stent void therein; and
suspending the coated stent within the stent void such that the coated stent is restricted from movement with respect to the tray when placed in the stent void.
